(19) United States
(12) Patent Application Publication
(10) Pub. No.: US 2017/0224924 A1
(43) Pub. Date: Aug. 10, 2017

(54) A Stop Mechanism For A Hypocycloid End-Of-Content Mechanism In An Injection Device

(71) Applicant: Novo Nordisk A/S, Bagsvaerd (DK)

(72) Inventor: Martin Johst Christensen, Copenhagen (DK)

(21) Appl. No.: 15/329,466

(22) PCT Filed: Jul. 27, 2015
(86) PCT No.: PCT/EP2015/067144

§ 371 Date: Jan. 26, 2017

(30) Foreign Application Priority Data
Jul. 28, 2014 (EP) 14178689.7

(51) Int. Cl. A61M 5/315; A61M 5/24; A61M 5/20

(52) U.S. Cl. CPC A61M 5/31541; A61M 5/3155; A61M 5/20; A61M 5/31511; A61M 5/24

(57) ABSTRACT

The invention relates to a stop mechanism for a non-axial working End-of-Content mechanism which is geared by a hypocycloid gearing. The EoC mechanism comprises a stationary first element (10), an EoC element (30) and a rotational element (50) with a cam surface. The EoC element rotates around a center axis which is dislocated in relation to the center axis of the first element. The EoC element thus works as a hypocycloid element which rotates through a specific angle whenever a rotational element driven by a dose setting button is rotated one full revolution. The EoC element thus counts the number of set doses.

### THE TECHNICAL FIELD OF THE INVENTION

The invention relates to a stop mechanism for an End-of-Content mechanism for an injection device. The invention specifically relates to such End-of-Content mechanism which operates without any axial movement thus making it suitable to be built into relatively short injection devices and especially to an EoC mechanism based on a hypocycloid gearing.

### DESCRIPTION OF RELATED ART

Injection devices for injecting an adjustable amount of a liquid drug usually have a button that a user rotates to set the adjustable size of the dose to be injected. Such injection devices holds a cartridge containing a specific amount of liquid drug and is usually equipped with a mechanism which secures that a user cannot set a dose size which exceeds the injectable amount remaining in cartridge at any time.

In mechanical injection devices this mechanism is usually some kind of counter which is moved whenever a dose is set but maintained in its new position when the dose is injected. The position of the counter is thus an expression of the accumulated doses set by the user. The movement of the counter is then restricted in accordance with the initial injectable quantum in the cartridge such that the counter is blocked in its movement when the accumulated doses set equals the initial injectable quantum in the cartridge.

Such mechanism is often referred to as an End-of-Content (EoC) mechanism and a very simple example is provided in U.S. Pat. No. 4,973,318. In this injection device the counter nut is formed integral with the dose setting button and is rotated up the threaded piston rod when a dose is set. When the set dose is injected, the counter nut is maintained in its relatively position on the thread of the piston rod as the dose setting button and the piston rod is moved axially forward. The length of the thread correlates to the initial injectable quantum of liquid drug in the cartridge and once the counter nut reaches the end of the thread no further dose can be set.

However, in this injection device the axial distance the injection button is moved during injection corresponds to the axial distance that the piston rod is moved forward inside the cartridge.

More modern injection devices has a gearing mechanism such that the piston rod can be moved a different length than the injection button is moved. An End-of-Content mechanism for such modern injection devices is disclosed in U.S. RE41.956.

FIG. 3 of U.S. RE41.956 discloses an embodiment in which a counter nut is moved up a helical track on a driver whenever a dose setting member is rotated. During injection, the counter nut is maintained in its relative position in the helical track such that the position of the counter nut in the helical track at any time is an expression of the accumulated doses set by the user. The length of the helical track correlates to the initial injectable quantum of liquid drug in the cartridge and once the counter nut reaches the end of the helical track, the dose setting member cannot be rotated further thus a dose larger than what corresponds to the length of the helical track cannot be set.

FIG. 2 of U.S. RE41.956 discloses a different embodiment wherein the End-of-Content mechanism is non-axial working. Here the driver is provided with a spiral track and the dose setting member is provided with a track follower engaging the track. The track and the track follower is rotated relatively to each other during dose setting but maintained in a relatively fixed position during injection. Once the spiral track ends, the track follower and thus the dose setting member cannot be moved further. However, since the length of the spiral track has to correlate to the initial injectable quantum of drug in the cartridge, the driver need to have a rather large diameter which disqualifies the use of this type of EoC mechanism in pen shaped injection devices.

An injection device similar to one disclose in U.S. RE41.956 is disclosed in WO 2013/170392. The injection device disclosed in WO 2013/170392 has a dose setting button which travels axially both during dose setting and during expelling of the set dose. Internally this dose setting button is provided with an End-of-Content mechanism which thus also travels axially both during dose setting and during expelling of the set dose. The End-of-Content mechanism disclosed in this document is based on a planetary gear mechanism having a planetary element that rotates around its own axis by a rotation of an outer element. After the planetary element has rotated several times around its own axis the planetary element encounters a stop hindering further dose setting.

A similar rotational stop mechanism limiting the number of revolutions of a shaft is disclosed in U.S. Pat. No. 3,411,366.

A different End-of-Content mechanism is disclosed in EP 1,861,141. In this EoC mechanism a first rotatable element rotates a second rotatable element one increment for each full rotation of the first element. A mechanism is provided which moves the second element axially in relation to the first element such that the two elements only engages and rotate together once for each full rotation of the first element. Once the second element has been rotated a specific and predetermined number of times the second element is arrested by a stop means and thus prevents both the second element and the first element from being rotated further. However, the axial movement of the second rotatable element in and out of its engagement with the first element requires some axial space inside the injection device.

In the recent years automatic spring driven injection devices have become very popular. These injection devices has a spring, often a torsion spring, which is strained during dose setting and released to drive a piston rod forward during injection. Since the spring provides the force to drive the injection there is no need for the user to push an injection button back into the housing of the injection device during injection. These new injection devices therefore have no part which grows out from the housing during dose setting in order for a user to push the same part back into the housing during dose injection. As a result these new automatic injection devices have the same length all the time.

An example of an End-of-Content mechanism for such automatic injection device is disclosed in WO2007/017052. Here a helically movable counter nut is screwed up the thread on the threaded piston rod when a dose is set and maintained in its relative position during dose injection. Once the counter nut reaches the end of the thread on the piston rod, the counter nut prevents the dose setting member from being rotated any further which thereby prevents that a further dose in being set. The length of the thread on the piston rod correlates to the initial injectable amount of liquid drug in the cartridge such that the counter nut reaches the end of the track when the initial injectable quantum has been repetitive set.

A drawback for all these known End-of-Content mechanism is that they require either a substantial clear axial length of the injection device due to the axial working element or a relatively large diameter in order to carry the spiral track as in U.S. RE41.956 FIG. 2.

A torsion spring driven injection device having a hypocycloid geared End-of-Content mechanism is further described in WO 2014/117944 and a similar rotational stop mechanism limiting the number of revolutions of a shaft is disclosed in GB 862,641.

### DESCRIPTION OF THE INVENTION

It is an object of the present invention to provide an injection device in which the End-of-Content mechanism has no axial working component at all and which can easily be fitted into a pen-shaped injection device having an oblong shape and a limited diameter.

It is further an objective to provide such hypocycloid End-of-Content mechanism having a stable and reliable stop function.

The invention is defined in claim 1. Accordingly in one aspect the present invention relates to a mechanical counter mechanism which requires no axial movement for counting.

The hypocycloid End-of-Content mechanism according to claim 1 basically comprises three parts;
1. a stationary and non-rotatable first element having a first internal surface with a first internal diameter (D),
2. a rotational element having a cam surface, and
3. an EoC element having a second external surface with a second external diameter (d) which is smaller than the first internal diameter (D) and an internal surface rotationally abutting the cam surface.

The stationary first element has a first centre axis (X), and the EoC element has a second centre axis (Y) being dislocated or offset in relation to the first centre axis (X). The EoC element rotates on the cam surface of the rotational element when rotated such that the second external surface of the EoC element engages with the first internal surface of the first element.

In order to utilize the hypocycloid for counting rotations, the stationary first element on the first internal surface carries a plurality of first teeth separated by first valleys, and the EoC element on the second external surface carries a plurality of second teeth separated by second valleys. During rotation, the first and second teeth engage with the second and first valley. When the rotational element is rotated e.g. by rotating a dose setting button, the EoC element is forced to rotate due to the engagement with the cam surface. The angular rotation of the EoC element is thus used as a counter for counting the sum of set doses.

Further a stopping means for halting the EoC element in a predetermined position is provided. The stopping means comprises a first stopping surface and a second stopping which engages in a predetermined stop position. The first stopping surface is provided on the EoC element and the second surface is provided on the rotational element preferably as a part of the cam surface. Whenever the first and the second stopping surface abut in the predetermined stop position, further rotation of the EoC element is prevented.

Once the EoC element arrives at the predetermined stop position further means are provided to move the EoC element radially such that the first and the second stop surfaces abut and thus prevents further dose setting.

To force the first and the second stopping surface to abut, at least one tooth of the EoC element or the stationary first element extend longer than the remaining teeth in the plurality of teeth in an axial direction, and at least one valley in the plurality of first valleys or second valleys has a partly filled out volume such that the extended tooth engages the filled out volume when the EoC element enters into the predetermined position thereby moving the EoC element radially such that the first stopping surface and the second stopping surface abut in the predetermined stop position.

Since the teeth provided on the EoC element or inside the stationary element preferably extend in a longitudinal direction along a centre axis of the injection device, extending longer means longer in the longitudinal direction. In the same manner partly filled out means that a part of the valley seen in a longitudinal direction is filled out.

The abutment between the extended tooth on one of the EoC element or the first element and the filled out volume on the other of the EoC element or the first element forces the EoC element to move in a radial direction which makes the first and the second stopping surface abut in the predetermined stop position to thereby prevent further dose setting.

The EoC element can have any shape desired but is preferably formed as a circular ring having an outer surface abutting the internal surface of the stationary and non-rotational first element and an inner surface abutting the cam surface of the rotational element.

Rotation of the rotational element in one rotational direction imparts a hypocycloidal rotation of the EoC element in the opposite rotational direction. Is the rotational element e.g. rotated clock-wise, the EoC element is forced to rotate in the anti-clock wise direction.

The hypocycloidal rotation of the EoC element in the opposite rotational direction also means that the EoC element only travels a fraction of the degrees the rotational element rotates. In one specific example of the invention, the stationary element has 24 teeth and valleys and the EoC element has 23 teeth and valleys. The teeth and the diameter ratio are such that one specific tooth on the EoC element moves one valley by each full rotation of the rotational element. The movement of the specific tooth on the EoC element thus counts the numbers on full rotations of the rotational element i.e. counting one step for each full rotation and this counting is correlated to the amount of injectable drug in the injection device such that the first and the second stopping surface abuts when the summarized amount set equals the initial injectable content liquid drug in the injection device.

The cam surface upon which the EoC element travels has an eccentric shape and preferably an elliptic shape. The eccentric shape of the cam surface is such that when the rotational element is rotated around the center axis (X) of the stationary element which is also the centre axis of the injection device it imposes a rotation of the offset EoC element which during its rotation move along the inner surface of the stationary element.

Further, in order to facilitate a proper engagement of the first stopping surface and the second stopping surface, a part of the cam surface provided on the rotational element can in one example be formed as a flexible arm which is bended when the EoC element is forced to move radially by the engagement between the prolonged tooth and the partly filled out volume.

In a specific example of the invention, the prolonged tooth is provided on the EoC element and the partly filled out volume is provided between two valleys of the stationary element.

The non-axial working limiting mechanism is preferably for use in a pen-shaped injection device in which the liquid drug is contained in a cartridge permanently embedded in the injection device i.e. in a so-called pre-filled injection device. Such injection devices are characterized in being delivered to a user with a fixed volume of liquid drug and being discharged by the user when that fixed content has been ejected through a number of individually set doses.

Should the described End-of-Content mechanism be used in a re-usable injection device it would require an additional mechanism to re-set the End-of-Content mechanism since it would be necessary to move the End-of-Content counter back to its initial position whenever a new and fresh cartridge is inserted.

The injection device carrying the non-axial working limiting mechanism according to the invention preferably has the rotational element coupled to a dose setting button to rotate with the dose setting button during dose setting, and the stationary first element coupled to a housing forming the outer boundaries of the injection device.

A torsion spring is preferably operational encompassed between the housing and the rotational element such that the torsion spring is strained whenever a user rotates the dose setting button and the rotational element. The dose setting button is rotorical coupled to the housing and does not travel axially in relation to the housing during dose setting.

The rotational element is thus preferably coupled to the dose setting button to rotate with the dose setting button such that whenever a user dials a dose this is translated to a rotation of the rotational element and the stationary element is coupled to the housing or alternatively formed unitary with the housing.

In a preferred embodiment disclosed in WO 2014/001318, the rotational element is coupled to a drive tube which again is coupled to the torsion spring. When the set dose is ejected, the rotational element is decoupled from the drive tube, such that the strained torsion spring rotates the drive tube which is at least during dose ejection temporally coupled to a piston rod drive element which thus moves the piston rod in the distal direction thus expelling the set dose.

The EoC element is henceforth rotated whenever the dose setting button is rotated relatively to the housing such that the EoC element step by step counts and remembers the rotations of the dose setting member.

Definitions:

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

The term “Needle Cannula” is used to describe the actual conduit performing the penetration of the skin during injection. A needle cannula is usually made from a metallic material such as e.g. stainless steel and connected to a hub to form a complete injection needle also often referred to as a “needle assembly”. A needle cannula could however also be made from a polymeric material or a glass material. The hub also carries the connecting means for connecting the needle assembly to an injection apparatus and is usually moulded from a suitable thermoplastic material. The “connection means” could as examples be a luer coupling, a bayonet coupling, a threaded connection or any combination thereof e.g. a combination as described in EP 1,536,854.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

By the term “Pre-filled” injection device is meant an injection device in which the cartridge containing the liquid drug is permanently embedded in the injection device such that it cannot be removed without permanent destruction of the injection device. Once the pre-filled amount of liquid drug in the cartridge is used, the user normally discards the entire injection device. This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

“Scale drum” is meant to be a cylinder shaped element carrying indicia indicating the size of the selected dose to the user of the injection pen. The cylinder shaped element making up the scale drum can be either solid or hollow. “Indicia” is meant to incorporate any kind of printing or otherwise provided symbols e.g. engraved or adhered symbols. These symbols are preferably, but not exclusively, Arabian numbers from “0” to “9”. In a traditional injection pen configuration the indicia is viewable through a window provided in the housing.

Using the term “Automatic” in conjunction with injection device means that, the injection device is able to perform the injection without the user of the injection device delivering the force needed to expel the drug during dosing. The force is typically delivered—automatically—by an electric motor or by a spring drive. The spring for the spring drive is usually strained by the user during dose setting, however, such springs are usually prestrained in order to avoid problems of delivering very small doses. Alternatively, the spring can be fully preloaded by the manufacturer with a preload sufficient to empty the entire drug cartridge though a number of doses. Typically, the user activates a latch mechanism e.g. in the form of a button on, e.g. on the proximal end, of the injection device to release—fully or partially—the force accumulated in the spring when carrying out the injection.

The term “Permanently connected” as used in this description is intended to mean that the parts, which in this application is embodied as a cartridge and a needle assembly, requires the use of tools in order to be separated and should the parts be separated it would permanently damage at least one of the parts.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show an exploded view of the EoC mechanism.

FIG. 2 show a perspective view of the ratchet or rotational element.

FIG. 2a show a perspective view of the ratchet arm.

FIG. 3 show perspective view of the EoC or second element.

FIG. 4 show perspective view of the first element.

FIG. 5 show a cross section of the End-of-Content mechanism about to reach its predetermined locked position.

FIG. 6 show a cross section of the End-of-Content mechanism in its predetermined locked position.

FIG. 7 show the End-of-Content mechanism build into a torsion spring driven injection device.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

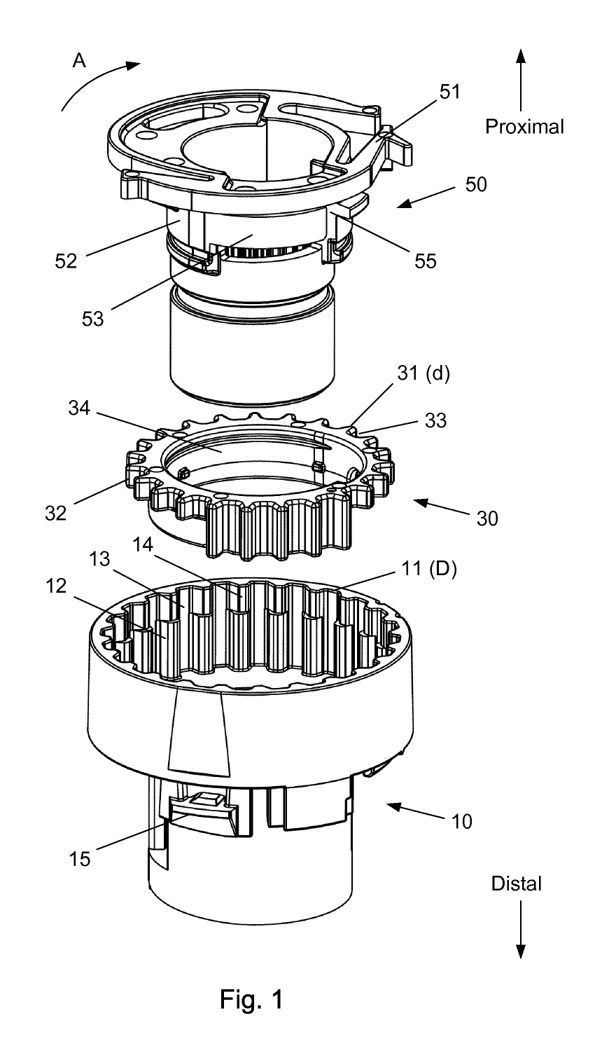

In that context it may be convenient to define that the term “distal end” in the appended figures is meant to refer to the end of the injection device which usually carries the injection needle whereas the term “proximal end” is meant to refer to the opposite end pointing away from the injection needle and usually carrying the dose dial button. In the figures, the rotational element 50 (the ratchet element) is provided at the proximal end as indicated in FIG. 1.

The hypocycloid End-of-Content mechanism basically comprises three elements shown in FIG. 1.
A first element 10.
A second element 30 also referred to in this text as the EoC element or EoC ring.
A third element 50 also referred to as the rotational element.

The first element 10 has a first internal surface 11 having a first diameter (D). This first surface 11 is provided with a number of inwardly pointing first teeth 12 which are separated by a number of first valleys 13. Proximally these first teeth 12 has a different radial height as can also be seen in FIG. 4. This proximal part 14 of the teeth 12 engages the ratchet arms 51 of the rotational element 50 as will be explained later.

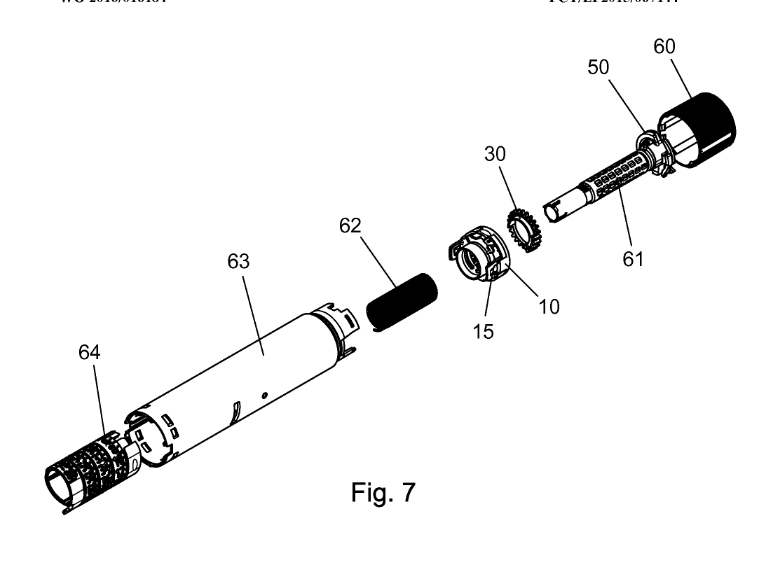

This first element 10 is stationary in relation to an outer frame of an injection device. The outer frame is usually the housing 63 of an injection device, and the first element 10 is either an integral part of the housing 63 or it is a separate part inrotatable secured to the housing 63 as disclosed in FIG. 7. In the disclosed example, the first element 10 is provided with protrusions 15 for securing the first element 10 to the housing 63 such that the first element can neither rotate nor move axially in relation to the housing 63.

The EoC element 30 has a second external surface 31 with a second external diameter (d). This second surface 31 is provided with a number of outwardly pointing second teeth 32 which are separated by a number of second valleys 33.

The second external diameter (d) of the external surface 31 is smaller than the internal first diameter (D) of the first element 10 and the EoC element 30 is thus able to rotate inside the first element 10.

Further, the EoC element 30 has an internal surface 34 which is guided on a cam surface 52 provided on the rotational element 50. The cam surface 52 is preferably elliptic and secures that the second teeth 32 of the EoC element 30 stays engaged with the first valleys 13 of the first element 10.

The stationary first internal surface 11 has a first centre line X as disclosed in FIG. 6, and the second external surface 31 has a second centre line Y. Due to the cam surface 52 of the rotational element 50, the second centre line Y is dislocated relatively to the centre line X as is known from a hypocycloid.

The rotational element 50 is coupled to a dose setting button 60 which is rotated by a user to set a desired dose to be injected. The rotational element 50 can be directly coupled to follow the rotation of the dose setting button 60, or it can be connected via a gearing such that the dose setting button 60 and the rotational element 50 rotate with different rotational velocities.

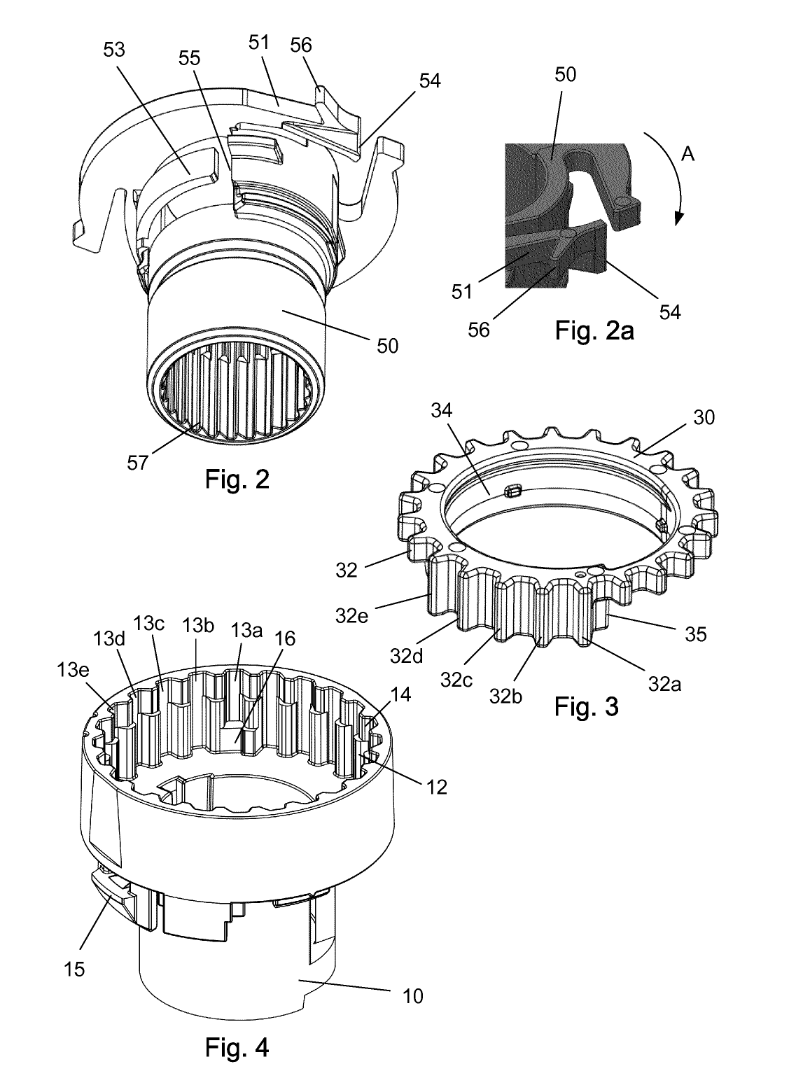

The rotational element 50 is provided with a radial ratchet arm 51 which engages the proximal part 14 of the teeth 12 of the first element 10. The lower part of the tip 54 of the ratchet arm 51 shown in details in FIG. 2a breaks against a flange on the teeth 12 such that the ratchet arm 51 is only allowed to rotate clock-wise in relation to the teeth 12 of the first element 10 and thus prevents rotation in the counter clock-wise direction. A mechanism for bending the ratchet arms 51 in a radial direction can easily be provided. When the ratchet arm 51 are moved radially to perform an inwardly pointing movement, the tip 54 of the arm 51 slip out of the engagement with the teeth 12 which allow the rotational element 50 to also rotate in the counter clock-wise direction. The ratchet arm 51 could e.g. be moved radially by activating the ratchet arm 51 itself, e.g. as disclosed in WO 2013/178372, or alternatively by activating an outward pointing part 56 of the ratchet arm 51 to move the ratchet arm 51 radially.

When the rotational element 50 is rotated in the clock-wise direction (A) by the dose setting button and the first element 10 is kept static, cam surface 52 upon which the EoC element 30 rotates forces the EoC element 30 to rotate counter clock-wise (indicated by the arrow “B”).

In the depicted embodiment, the first element 10 has a number of 24 valleys 13 equally distributed over the first internal diameter (D) and the EoC element 30 has a number of 23 teeth 32 equally distributed over the second external diameter (d).

The diameter ratio (D/d) and thereby the ratio of valleys 13, 33 and teeth 12, 32 are calculated such that the first tooth 32a on the EoC element 30 shifts to the next consecutive (anti clock-wise) valley 13 of the first element 10 whenever the rotational element 50 is rotated one full rotation (i.e. 360 degrees) clock-wise.

The hypocycloid gearing, which is further explained in WO 2014/117944 is thus configured such that the first tooth 32a is moved 15 degrees (360/24) counter clock-wise (B) whenever the rotational element 50 is rotated 360 degrees clock-wise (A). The EoC element 30 thus rotates 15 degrees in relation to the first element 10 for each full rotation of the rotational element 50.

A number of the teeth 32 on the EoC element 30 are prolonged in the axial direction of the injection device. The number of prolonged teeth 32a, b, c, d, e can be any number but in the depicted embodiment a total number of 5 of the teeth 32 are prolonged.

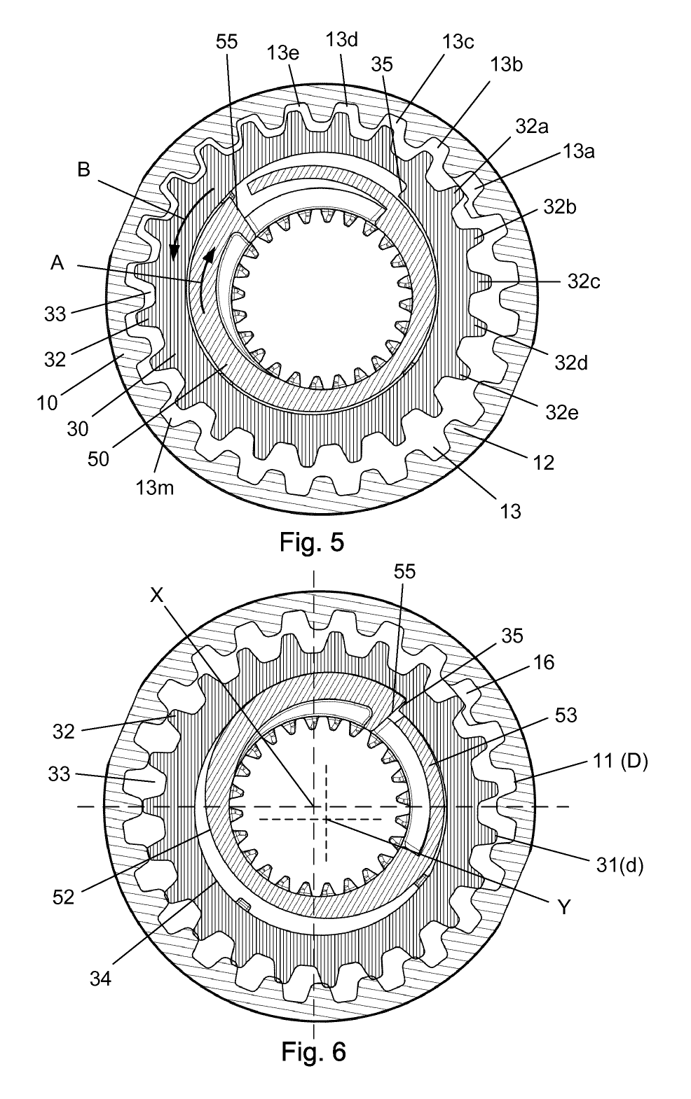

The working mode will be explained in relation to the engagement between the first tooth 32a of the EoC element 30 and the first valley 13a of the first element 10 as disclosed in FIG. 5 and FIG. 6. The prolonged teeth 32 following the first tooth 32a clock-wise are marked respectively 32b, 32c, 32d and 32e. In the same manner, the valleys 13 laying adjacent to the first valley 13a in the counter clock-wise direction is marked 13b, 13c, 13d and 13e respectively.

The total height of the EoC element 30 (in an axial direction) are approximately the same as the height of the prolonged teeth 32a-e which again has the same height as the distal part of the teeth 12 of the first element 10 such that the EoC element 30 rotate inside the distal part of the first element 10.

As previously explained, the ratchet arm 51 of the rotational element 50 engages the upper part 14 of the teeth 12 of the first element 10.

In order to interact with one of the prolonged teeth 32a-e, one specific valley 13a (dedicated as the first) of the first element 10 has a partly filled out volume 16 in the part able to come into contact with the prolonged part of one prolonged teeth 32a-e.

Whenever, the first prolonged tooth 32a engages the filled out volume 16 of the first valley 13a (arriving counter clock-wise (B)), the EoC element 30 is pushed out of its rotational engagement and jams. As a result the EoC ring 30 is unable to rotate any further as depicted in FIG. 6, thus the EoC ring 30 is only able to rotate less than 306 degrees.

Further, the EoC element 30 is provided with a first stop surface 35 which engages a second stop surface 55 on the rotational element 50. These two stopping surfaces 35, 55 are provided such that they engage each other when the EoC ring 30 jams as depicted in FIG. 6.

The part of the rotational element 50 clock-wise adjacent to the second stop surface 55 is made as a flexible arm 52 which can deflect when the two stopping surfaces 35, 55 engages.

In a new and fully filled injection device, the last (=the fifth) 32e of the prolonged teeth 32a-e is located in the second valley 13b counter clock-wise of the filled out valley 13a. This is, so to speak, the start position for a new and fresh injection device. In the depicted example this allows the first prolonged tooth 32a to move through a number of 18 free valleys 32 (23 free valleys minus 5 prolonged teeth=18) before it encounters the first valley 13a which has the filled volume 16. This therefore allows the rotational element 50 to rotate (18+1=) 19 full rotations before the EoC ring 30 jams. The nineteen's revolution is the very last full rotation stretching from the last of the free valleys until the tooth 32a encounters the partly filled out valley 13a.

If the injection device is a typical insulin injection device it would e.g. be filled with 3.0 ml of U100 insulin which would amount to a total of 300 I.U. Usually such injections devices have a dose dial of 24 I.U per full revolution. Thus the requirement before the 300 I.U is ejected is (300/24) 12.5 full revolutions of the rotational element 50. The EoC element 30 would then during manufacture of the injection device just need to be placed such that the first prolonged tooth 32a is in the correct starting valley 13 such that the first prolonged tooth 32a enters the filled out valley 13a when the rotational element 50 has been rotated 12.5 times.

The half rotation is best obtained by having a rotational distance of 180 degrees (i.e. half of a full rotation) between the first stop surface 35 and the second stop surface 55 in the start position. The rotational element 50 would then be allowed to move 12 full rotations before the first prolonged tooth 32a start to engage the filled out volume 16 of the first valley 13a and due to the 180 degrees angular different start position of the first stop surface 35 and the second stop surface 55 half a rotation will be added to the 12 full rotations thus allowing the rotational element 50 to rotate 12.5 times 360 degrees. The valley 13 in which the first tooth 32a should start is indicated 13m in FIG. 5.

It should thus be clear that if e.g. a U200 insulin is used, the 3.0 ml would contain 600 I.U and with 24 International Units per revolution, it would require the first tooth 32a to move through 24 free valleys 13 and into the partly filled-out twenty-fifth valley (=25 full rotations). It would thus require a different number of teeth 12, 32 and valleys 13, 33 which would still be within the invention as claimed in the appended claims.

During assembly of the drive mechanism for the injection device the first tooth 32a can be positioned in a different location than in the above examples. In some cases the torsion spring of the device is pre-tensioned during assembly meaning that the tooth 32a might be rotated counter clock-wise during assembly. In a different example, the drive mechanism could be tested after assembly but before assembling the entire injection device. In this case the tooth 32a might move clock-wise during assembly. However, this can easily be incorporated into the starting position of the tooth 32a. Once the injection device is finally assembled and delivered to the user, the tooth 32a has to be able to move the relevant amount of valleys 13 before reaching the filled out 16 valley 13a.

In order to allow the first stop surface 35 to properly engage with the second stop surface 55, a part of the cam surface 52 is provided on a flexible arm 53 which is allowed to flex as disclosed in FIG. 6, thus allowing the first surface 35 to abut the second surface 55.

FIG. 7 discloses the End-of-Content mechanism build into a torsion spring operated injection device. Proximally a dose setting button 60 is provided which engages the rotational element 50 such that the rotational element 50 rotate in the direction “A” whenever the dose setting button 60 is rotated to set a dose.

Internally the dose setting button 60 is provided with a protrusion like part which is able to bend the ratchet arm 51 radially when the dose setting button 60 is rotated in the opposite direction to lower a set dose.

The rotational element 50 is internally provide with a toothing 57 which couples the rotational element 50 to a ratchet element 61 which thereby rotate together with the rotational element 50. The ratchet element 61 could alternatively be moulded together with the rotational part 50 to form one unitary unit.

The ratchet element 61 is coupled to a not-shown drive element which is rotated together with the ratchet element 61 during dose setting. This is disclosed in details in WO 2014/001318 (see especially FIG. 20).

A torsion spring 62 is operational between a housing 63 and the drive element such that the torsion spring 62 is strained whenever the ratchet element 61 is rotated by the rotational element 50.

The torque build up in the torsion spring 62 during dose setting is held by the engagement between the ratchet arm 51 and the proximal part 14 of the teeth 12 of the first element 10 and can be released by moving the ratchet element 61 and the drive element out of engagement such that the torque of the torsion spring rotates the drive element.

The dose set is visualized to the user by a rotatable scale drum 64 provided between the drive element and the housing 63. This scale drum 64 is externally provided with a helical track which engages a similar track provided inside the housing 63 to move the scale drum 64 helically both during dose setting and during expelling of the set dose.

Some preferred embodiments have been shown in the foregoing, but it should be stressed that the invention is not limited to these, but may be embodied in other ways within the subject matter defined in the following claims. It is especially stressed that the described hypocycloid geared EoC mechanism can easily be adjusted to accommodate any size of dosing from any initial content of liquid drug. It is further stressed that the disclosed positions of the EoC mechanism in the described embodiments could be different. The EoC mechanism could e.g. be provided in a different injection device and e.g. in a different position in the injection device itself.

1. A non-axial working limiting mechanism for an injection device which prevents setting of a dose exceeding the injectable amount of liquid drug contained in the injection device, the limiting mechanism comprising;
a stationary and non-rotatable first element having a first internal surface with a first internal diameter (D),
a rotational element having a cam surface,
an EoC element having a second external surface with a second external diameter (d) being smaller that the first internal diameter (D) and an internal surface rotationally abutting the cam surface and which EoC element is at least partly located inside the first internal surface of the first element, wherein;
the stationary first element has a first centre axis (X), and the EoC element has a second centre axis (Y) being dislocated in relation to the first centre axis (X) such that the second external surface of the EoC element engages with the first internal surface of the first element,
the stationary first element on the first internal surface carries a plurality of first teeth separated by first valleys, and the EoC element on the second external surface carries a plurality of second teeth separated by second valleys and wherein the first and second teeth engages with the second and first valleys, and
the EoC element is provided with a first stopping surface and the rotational element is provided with a second stopping surface such that further rotation of the EoC element is prevented when the two surfaces abut in a predetermined stop position, and wherein
means are provided moving the EoC element radially such that the first stopping surface and the second stopping surface abut when the EoC element enters into the predetermined stop position.

2. A non-axial working End-of-Content mechanism according to claim 1, wherein the means for moving the EoC element radially, comprises:
a partly filled out volume of one valley in the plurality of first valleys or second valleys and
at least one tooth of the EoC element or the stationary first element extending longer than the remaining teeth in the plurality of teeth in an axial direction, such that the extended tooth engages the filled out volume in the predetermined stop position.

3. A non-axial working End-of-Content mechanism according to claim 1, wherein the EoC element is ring shaped.

4. A non-axial working End-of-Content mechanism according to claim 1, wherein the EoC element rotate in a rotational direction opposite to the rotational direction of the rotational element.

5. A non-axial working End-of-Content mechanism according to claim 1, wherein the cam surface has an elliptic shape.

6. A non-axial working End-of-Content mechanism according to claim 1, wherein a part of the cam surface on the rotational element is formed as a flexible arm.

7. A non-axial working End-of-Content mechanism according to claim 2, wherein one or more of the teeth provided on the EoC element is prolonged.

8. A non-axial working End-of-Content mechanism according to claim 2, wherein at least one of the valleys of the stationary first element has a filled out volume.

9. An injection device for apportioning set doses of a liquid drug, comprising:
a non-axial working limiting mechanism which prevents setting of a dose exceeding the injectable amount of liquid drug contained in the injection device according to claim 1,
wherein the rotational element is coupled to a dose setting button to rotate with the dose setting button at least during dose setting, and the stationary first element is coupled to a housing forming the outer boundaries of the injection device.

10. An injection device according to claim 9, wherein a torsion spring is operational encompassed between the housing and the rotational element.

11. An injection device according to claim 9, wherein the dose setting button does not travel axially in relation to the housing during dose setting.

12. An injection device according to claim 9, wherein the dose setting button and the rotational element rotate around the same longitudinal extending centre axis (X) during dose setting.
